(19) United States
(12) Patent Application Publication
(10) Pub. No.: US 2019/0343871 A1
(43) Pub. Date: Nov. 14, 2019

(54) Compositions and Methods for Inhibiting Shear Induced Platelet Accumulation

(71) Applicant: Georgia Tech Research Corporation, Atlanta, GA (US)

(72) Inventors: Michael T. Griffin, Atlanta, GA (US); Cyrus K. Aidun, Marietta, GA (US); David N. Ku, Decatur, GA (US)

(21) Appl. No.: 16/480,877

(22) PCT Filed: Jan. 31, 2018
(86) PCT No.: PCT/US2018/016165

§ 371 Date: Jul. 25, 2019

Related U.S. Application Data
(60) Provisional application No. 62/452,729, filed on Jan. 31, 2017.

(51) Int. Cl. A61K 31/765; A61K 9/14

(52) U.S. Cl. CPC A61K 31/765; A61K 9/14

(57) ABSTRACT

Methods of modulating the conformation of vWF are provided. One embodiment provides a method for linearizing globular vWF by contacting globular vWF proteins with an effective amount of negatively charged nanoparticles that interact with the globular vWF proteins to induce a conformational change in the globular vWF proteins such that the change in conformation inhibits or reduces the ability of the vWF proteins to bind to platelet receptors. Preferred negatively charged nanoparticles are non-functionalized.

### CROSS-REFERENCE TO RELATED APPLICATIONS

This application claims benefit of and priority to U.S. Provisional Patent Application No. 62/452,729 filed on Jan. 31, 2017, and where permitted is incorporated by reference in its entirety.

### TECHNICAL FIELD OF THE INVENTION

Aspects of the invention are generally directed to nanoparticle compositions for inhibiting shear-induced platelet deformation.

### BACKGROUND OF THE INVENTION

Current antiplatelet therapies, such as aspirin (ASA) and Plavix® (Clopidogrel), are designed to inhibit platelet activation and binding via irreversible biochemical means. However, these drugs do not work as intended for the entire population, as 5-50% of patients exhibit an “antiplatelet resistance” under recommended doses (Gum, P. A., et al., J Am Coll Cardiol, 41(6):961-5 (2003); Hovens, M. M., et al., Am Heart J,. 153(2):175-81 (2007); Michos, E. D., et al., Mayo Clin Proc,. 81(4): p. 518-26 (2006)).

The observed resistances to leading antiplatelet agents prove the need to research and develop novel therapeutic agents. Recently, efforts to increase drug delivery efficiency have been made through drug-functionalized nanoparticles (Cicha, I., World J Cardiol, 7(8):434-41(2015)). Through conjugating ASA to peptide-based nanoparticles, an increase in COX-1 inhibition was observed within a rat model (Chen, Y., et al., Bioorg Med Chem, 16(11):5914-25 (2008); Jin, S., et al., ACS Nano, 7(9):7664-73 (2013)). Related work on inhibiting stent thrombosis has made use of PPACK conjugated perfluorocarbon nanoparticles to inhibit thrombin (Palekar, R. U., et al., J Vasc Surg, 64(5): 1459-1467(2016)). Another particle has been designed to take advantage of the high shear flow at the site of an arterial thrombus to locally release the thrombolytic agent tPA (Korin, N., et al., JAMA Neurol,. 72(1): p. 119-22. However, no such functionalized particle has been developed to fully alleviate non-responsive therapies.

Because of the increase of nanomaterials in medical and industrial applications, many investigations on the pathological cardiovascular consequences of particles have been undertaken. Particles are often found in environmental air samples and are thought to be toxic. Positive non-functionalized particles have been found to have strong effects on both cytotoxicity and vascular toxicity. However, negatively charged non-functionalized particles have low genotoxicity or cytotoxicity (Platel, et al 2016).

Thus, it is an object of the invention to provide polymeric nanoparticles that affect blood clotting by physical means, not pharmacological means.

### SUMMARY OF THE INVENTION

It has been discovered that small, charged particles can interact with the protein von Willebrand factor (vWF) and induce a conformational change in elongated vWF in shear flow that reduces or inhibits the ability of vWF to interact with platelets. One embodiment provides a method for inhibiting or reducing the bioactivity of vWF in a subject in need thereof by administering to the subject an effective amount of negatively charged nanoparticles that interact with vWF in the subject's circulatory system to induce a three-dimensional conformational change in the vWF proteins which in turn inhibits or reduces the interaction between the vWF proteins and platelets in the circulatory system of the subject. In one embodiment, the nanoparticles have an average diameter of about 10 to 1000 nm, more precisely between 25 and 300 nm, or a mixture of nanoparticles having individual diameters between 25 and 300 nm.

In one embodiment the nanoparticles are made of a negatively charged material. Negatively charged material means negatively charged under physiological conditions. The negatively charged particles have a unique ability to affect the thrombotic behavior of blood. When mixed or injected into whole blood, the negatively charged, small particles reduce the normal propensity of blood to form a clot. As myocardial infarction (heart attack) and cerebrovascular accident (stroke) is caused by acute thrombosis or clotting of an artery, the particles have the ability to reduce the incidence of the fatal clots. The particles act by electrostatic interaction with the vWF to prevent myocardial infarction without the usual pharmaceutical interactions of drugs. Since the particles do not have pharmaceutical effects, the toxicity is also reduced. Thus, the negatively charged, small molecules represents a new class of therapies for this widespread disease.

The nanoparticles can be formed of a biodegradable polymer, for example poly(lactic-co-glycolic acid) also referred to as PLGA. In other embodiments, the negatively charged nanoparticles are formed of a blend of two or more different polymers that form a negatively charged nanoparticle. In some embodiments the negatively charged nanoparticles are non-functionalized. For example the nanoparticles are not functionalized with a protein, lipid, therapeutic agent, or small molecule.

In certain embodiments, the negatively charged nanoparticles have a charge of about −1 to −500 mV. Preferably, the charge on the particles is between −25 to −80 mV.

In another embodiment, the negatively charged nanoparticles bind to the A1 domain of vWF and inhibit or reduce the binding of vWF to platelet receptors.

One embodiment provides a method for reducing or inhibiting shear-induced platelet accumulation in a subject in need thereof by administering to the subject an effective amount of negatively charged, biodegradable nanoparticles having an average diameter between 10 to 1000 nm and a charge of −1 mV to −500 mV to bind to the A1 domain of vWF proteins in the circulatory system of the subject and inhibit or reduce binding of nanoparticle-bound-vWF proteins to platelets in the subject's circulatory system. In one embodiment, the binding of nanoparticles to the vWF proteins is electrostatic binding due to the negative charge of the nanoparticles and substantially positive charge of the A1 domain. In another embodiment the shear-induced platelet accumulation occurs in an artery or vein of the subject.

In one embodiment, the negatively charged nanoparticles interact with the elongated vWF proteins to cause the vWF proteins to substantially return to globular conformation. The substantially globular vWF receptors cannot bind to platelet receptors including, but not limited to Glycoprotein Ib and Glycoprotein IIb/IIIa (Integrin α_{IIb}β₃). In one embodiment, the shear rate is in the range of 2,000 to 10,000 1/s, where the critical shear rate to substantially elongate the vWF is about 5000 1/s. The nanoparticle volumetric concentration is from 0.01 to 0.10%, the size range is 10 to 1000 nm, shear rate 2,000 to 10,000 1/s, and the vWF length when fully elongated is from 0.01 to 0.5 mm.

### BRIEF DESCRIPTION OF THE DRAWINGS

FIG. 1 is a graph showing the effect of PLGA and PS nanoparticle dosing on shear-induced platelet accumulation occlusion time within an in vitro microfluidic assay.

FIG. 2 is a quantitative imaging assessment of the level of shear-induced platelet accumulation in a control vs. nanoparticle whole blood in vitro microfluidic assay.

FIG. 3 is a computational model of equal time interval snapshots of conformational dynamics of a single vWF with charged nanoparticles and without charged nanoparticles in simple shear flow at 6000 1/s. FIG. 3A shows equal time interval snapshots of conformational dynamics of a single vWF with charged nanoparticles (dot, left) and without charged nanoparticles (right) in simple shear flow at 6000 1/s. Time, t, is scaled with the particle diffusion time, □. The charge of the nanoparticle is −50 mV; each monomer of vWF has +150 mV charge (same as the A1 domain), and there are 50 monomers in vWF. Note that the presence of 2 charged nanoparticles on the left prevents the elongation of the green vWF, where the VWF on the right. Without the charged particles elongates after initially folding. FIG. 3B shows the rapid collapse from scaled time 8.34 to 16.69 shown in smaller time intervals.

### DETAILED DESCRIPTION OF THE INVENTION

### I. Definitions

The term “shear-induced platelet accumulation” refers to the occurrence of platelet accumulation without the prior initiating step of platelet activation.

The term “negatively charged nanoparticle” refers to a particle of the charge −1 to −500 mV by zeta potential.

The term “non-functionalized nanoparticle” refers to particle that does not have a pharmaceutical agent, protein, lipid, therapeutic agent, or small molecule connected to the nanoparticle.

### II. Methods for Modulating vWF Conformation

Methods of modulating the conformation of vWF are provided. One embodiment provides a method for substantially globularizing of elongated vWF by contacting elongated vWF proteins with an effective amount of negatively charged nanoparticles that interact with the elongated vWF proteins under shear to induce a conformational change in the elongated vWF proteins such that the change in conformation inhibits or reduces the ability of the vWF proteins to bind to platelet receptors. Preferred negatively charged nanoparticles are non-functionalized.

### A. Negatively Charged Nanoparticles

### 1. Polymeric Nanoparticles

Exemplary nanoparticles that can be used to modulate the conformation of vWF proteins are made of a polymer or a blend of polymers. In one embodiment the polymer or polymer blend is biodegradable. Exemplary polymers include, but are not limited to biocompatible aliphatic polyesters such as PLGA, polylactic acid (PLA), polyalkylcyanoacrylate (PACA), polyvinylpyrrolidone (PVP), polymethylmethacrylate (PMMA), polymethylacrylate (PMA), polyhydroxyalkanoate (PHA), poly(glycerol sebacate), or copolymers or derivatives including these and/or other polymers.

In some embodiments, the polymers are biodegradable.

The term “polymer,” as used herein, is given its ordinary meaning as used in the art, i.e., a molecular structure comprising one or more repeat units (monomers), connected by covalent bonds. The repeat units may all be identical, or in some cases, there may be more than one type of repeat unit present within the polymer. In some cases, the polymer can be biologically derived, i.e., a biopolymer. It is to be understood that in any embodiment employing a polymer, the polymer being employed may be a copolymer in some cases. The repeat units forming the copolymer may be arranged in any fashion. For example, the repeat units may be arranged in a random order, in an alternating order, or as a block copolymer, i.e., containing one or more regions each comprising a first repeat unit (e.g., a first block), and one or more regions each containing a second repeat unit (e.g., a second block), etc. Block copolymers may have two (a diblock copolymer), three (a triblock copolymer), or more numbers of distinct blocks.

The disclosed nanoparticles can include copolymers, which, in some embodiments, describes two or more polymers (such as those described herein) that have been associated with each other, usually by covalent bonding of the two or more polymers together. Thus, a copolymer may comprise a first polymer and a second polymer, which have been conjugated together to form a block copolymer where the first polymer can be a first block of the block copolymer and the second polymer can be a second block of the block copolymer. Of course, those of ordinary skill in the art will understand that a block copolymer may, in some cases, contain multiple blocks of polymer, and that a “block copolymer,” as used herein, is not limited to only block copolymers having only a single first block and a single second block. For instance, a block copolymer may comprise a first block comprising a first polymer, a second block comprising a second polymer, and a third block comprising a third polymer or the first polymer, etc. In some cases, block copolymers can contain any number of first blocks of a first polymer and second blocks of a second polymer (and in certain cases, third blocks, fourth blocks, etc.). In addition, it should be noted that block copolymers can also be formed, in some instances, from other block copolymers. For example, a first block copolymer may be conjugated to another polymer (which may be a homopolymer, a biopolymer, another block copolymer, etc.), to form a new block copolymer containing multiple types of blocks, and/or to other moieties (e.g., to nonpolymeric moieties).

In one set of embodiments, a polymer (e.g., copolymer, e.g., block copolymer) contemplated herein includes a biocompatible polymer, i.e., the polymer that does not typically induce an adverse response when inserted or injected into a living subject, for example, without significant inflammation and/or acute rejection of the polymer by the immune system, for instance, via a T-cell response. Accordingly, the disclosed nanoparticles contemplated herein can be non-immunogenic. The term non-immunogenic as used herein refers to endogenous growth factor in its native state which normally elicits no, or only minimal levels of, circulating antibodies, T-cells, or reactive immune cells, and which normally does not elicit in the individual an immune response against itself.

Biocompatibility typically refers to the acute rejection of material by at least a portion of the immune system, i.e., a nonbiocompatible material implanted into a subject provokes an immune response in the subject that can be severe enough such that the rejection of the material by the immune system cannot be adequately controlled, and often is of a degree such that the material must be removed from the subject. One simple test to determine biocompatibility can be to expose a polymer to cells in vitro; biocompatible polymers are polymers that typically will not result in significant cell death at moderate concentrations, e.g., at concentrations of 50 micrograms/10⁶ cells. For instance, a biocompatible polymer may cause less than about 20% cell death when exposed to cells such as fibroblasts or epithelial cells, even if phagocytosed or otherwise uptaken by such cells. Another biocompatibility test is to expose culture cells to the test material and observe if there are changes or mutations in the genes (DNA) of the cells (genotoxicity). Non-limiting examples of biocompatible polymers that may be useful in various embodiments include poly(lactic-co-glycolic acid) (PLGA), polylactic acid (PLA), polyalkylcyanoacrylate (PACA), polyvinylpyrrolidone (PVP), polymethylmethacrylate (PMMA), polymethylacrylate (PMA), polyhydroxyalkanoate (PHA), poly(glycerol sebacate), or copolymers or derivatives including these and/or other polymers.

In certain embodiments, contemplated biocompatible polymers may be biodegradable, i.e., the polymer is able to degrade, chemically and/or biologically, within a physiological environment, such as within the body. As used herein, “biodegradable” polymers are those that, when introduced into cells, are broken down by the cellular machinery (biologically degradable) and/or by a chemical process, such as hydrolysis, (chemically degradable) into components that the cells can either reuse or dispose of without significant toxic effect on the cells. In one embodiment, the biodegradable polymer and their degradation byproducts can be biocompatible.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers containing glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids.

In some embodiments, a polymer may be PLGA. PLGA is a biocompatible and biodegradable co-polymer of lactic acid and glycolic acid, and various forms of PLGA can be characterized by the ratio of lactic acid:glycolic acid. Lactic acid can be L-lactic acid, D-lactic acid, or D,L-lactic acid. The degradation rate of PLGA can be adjusted by altering the lactic acid-glycolic acid ratio. In some embodiments, PLGA to be used in accordance with the present invention can be characterized by a lactic acid:glycolic acid ratio of approximately 85:15, approximately 75:25, approximately 60:40, approximately 50:50, approximately 40:60, approximately 25:75, or approximately 15:85. In some embodiments, the ratio of lactic acid to glycolic acid monomers in the polymer of the particle (e.g., the PLGA block copolymer), may be selected to optimize for various parameters such as water uptake, and/or polymer degradation kinetics can be optimized.

### 2. Negative Charge

In certain embodiments, the negatively charged, polymeric nanoparticles have a charge or zeta potential of about −1 to −500 mV or about −25 to −80 mV. The zeta potential can be measured using techniques known in the art. One measurement technique uses electrophoretic light scattering.

Devices for measuring zeta potential of nanoparticles are commercially available. An exemplary device is the Malvern ZetaSizer Nano™.

### 3. Size

The negatively charged nanoparticle can individually have a diameter of 10 to 1000 nm. In one embodiment, nanoparticle composition contains nanoparticles that have an average diameter of 10 to 1000 nm. Another embodiment provides a nanoparticle composition that contains nanoparticles having an average diameter of 25 to 300 nm.

### B. vWF

The disclosed negatively charged nanoparticles interact with vWF proteins in the circulatory system of a subject and alter the three-dimensional conformation of the vWF proteins and thereby inhibit or reduce the ability of the vWF proteins to bind to platelet receptors. The interaction of the nanoparticles can be non-covalent interaction such an electrostatic interaction. In one embodiment, the negatively charged nanoparticles bind to vWF proteins in the blood serum of a human subject.

vWF is a large multimeric glycoprotein in blood plasma and is involved in hemostasis. The basic vWF monomer is a 2050-amino acid protein. Every monomer contains a number of specific domains with a specific function. The amino acid sequence for human vWF is known in the art and has Uniprot accession number UniProtKB-P04275 (VWF_HUMAN), which is incorporated by reference in its entirety.

In quiescent fluid where there is no flow, VWF exists in a globular equilibrium state. The globular equilibrium state of VWF remains intact for shear rates of up about 5000 1/s. This is necessary for blood flow and hemostasis, as the normal and efficient mode of transport of vWF in blood is when it is in compact globular state. Furthermore, vWF consists of a string of monomers with ‘domains’, as outlined above, with specific but complex electrostatic surface charge. Without this structure and charge, vWF cannot function efficiently to capture platelets and form clots for hemostasis. The dynamics of collapsed VWF in the globular state is very different from that of elongated VWF in shear flow. The cohesive attraction between monomers has to give way to fluid shear stress before the monomer can elongate. Once the VWF elongates, in shear flow it will undergo a continuous conformational change from elongating to tumbling to folding where the average conformation is substantially in the elongated state.

One domain of vWF is the A1 domain which binds to: platelet GPIb-receptor, heparin, and possibly collagen. In one embodiment, the negatively charged nanoparticles interact with the A1 domain and induce a conformational change in the vWF proteins that inhibits or reduces binding of the vWF proteins to platelet receptors including but not limited to platelet GPIb-receptor. In one embodiment, the negatively charged nanoparticles globularize linear vWF proteins in blood plasma of human subject at shear rate of 6000 1/s. In general, 1 to 3 or more charged nanoparticles bind to the VWF and cause the linear VWF to substantially collapse to globular state at shear rate above the critical value. The charged particles may also prevent vWF binding to vWF or prevent the formation of vWF nets (Casa, et al 2015). In these cases the natural formation of thrombosis at high shear rates is hindered and myocardial infarctions may be reduced.

### C. Formulations and Administration

Nanoparticles disclosed herein may be combined with pharmaceutical acceptable carriers to form a pharmaceutical composition. As would be appreciated by one of skill in this art, the carriers may be chosen based on the route of administration as described below. Parenteral administration is a preferred route of administration.

The pharmaceutical compositions can be administered to a patient by known parenteral routes. The term “patient or subject,” as used herein, refers to humans as well as non-humans, including, for example, mammals, birds, reptiles, amphibians, and fish. For instance, the non-humans may be mammals (e.g., a rodent, a mouse, a rat, a rabbit, a monkey, a dog, a cat, a primate, or a pig). In certain embodiments parenteral routes are desirable since they avoid contact with the digestive enzymes that are found in the alimentary canal. According to such embodiments, the pharmaceutical compositions may be administered by injection, preferably intravenous or intra-arterial injection.

In a particular embodiment, the nanoparticles are administered to a subject in need thereof systemically, e.g., by IV infusion or injection.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

### III. Methods of Treatment

One embodiment provides a method for altering tertiary length of vWF by administering to a subject in need thereof an effective amount of a the disclosed negatively charged nanoparticles or a pharmaceutical composition containing negatively charged, non-functionalized polymeric nanoparticles to interact or non-covalently with linear vWF to globularize the linear vWF in the subject, wherein the globular vWF proteins are under physical shear stress.

Another embodiment provides a method for extending or prolonging platelet occlusion time in a subject in need thereof by administering to the subject an effective amount of negatively charged, non-functionalized nanoparticles or a pharmaceutical composition thereof that binds to the A1 domain of vWF proteins in blood plasma of the subject to induce a tertiary conformational change in the vWF, wherein the induced conformational change inhibits or reduces binding of vWF to platelet receptors.

Still another embodiment provides a method for reducing or inhibiting myocardial infarction or stroke in a subject in need thereof by administering to the subject an effective amount of negatively charged, non-functionalized nanoparticles or a pharmaceutical composition thereof that binds to the A1 domain of vWF proteins in blood plasma of the subject to induce a tertiary conformational change in the vWF, wherein the induced conformational change inhibits or reduces binding of vWF to platelet receptors.

### EXAMPLES

### Example 1: PLGA Nanoparticles Reduce Occlusion Time

### Materials and Methods

Biodegradable PLGA nanoparticles of negative surface charge were fabricated in house by nanoprecipitation methods. 100 mg of RG503H Resomer® (Sigma Aldrich) was dissolved in an 85:15 acetone to ethanol mixture. The dissolution was performed over 5 minutes at 150 g stirring. Ultrapure water was added with continued stirring at 150 g for 3 hours to create a final concentration of 10 mg/mL. Carboxylated polystyrene (PS) nanoparticles of 60 nm size were also purchased at a concentration of 10 mg/mL (Bangs Laboratories).

Particles were stored at 4° C. until needed for characterization or whole blood treatment. Prior to use, the particles were sonicated for 15 minutes and vortexed for 10 seconds to evenly disperse with minimal agglomeration. Characterization of average diameter and zeta potential was performed by addition of 100 μL of the 10 mg/mL particle mixture to 900 μL of deionized water for a final test concentration of 1 mg/mL. Diameter was measured by dynamic light scattering, while zeta potentials were measured by photon correlation spectroscopy (ZetaSizer Nano-ZA, Malvern Instruments).

Porcine whole blood was obtained from a local abattoir (Holifield Farms, Covington, Ga.) and lightly heparinized at 3.5 USP units/mL. Blood was stored at room temperature on a rocker prior to testing. All testing was completed within 8 hours after collection.

Blood samples were treated with varying concentrations of particles. Estimations of the appropriate dose were made from the reported effective value of 500 μg/kg body weight in the hamster model of Nemmar, et al 2002. Assuming an average body weight of 100 g and 7 mL of blood in the systemic circulation of the animal, the systemic particle concentration for a 500 μg/kg dose is approximately 7 μg/mL blood. The concentration of 7 μg/mL was treated as the baseline concentration, with multiples of this concentration of 14, 21, 28, 35, and 70 μg/mL also investigated.

Microfluidic chips were created by casting PDMS (Sylgard 184, Krayden) on a custom-etched silicon 3D stenotic microfluidic mold. After plasma bonding the PDMS to 25×75 mm glass slide, the devices were coated with bovine type I fibrillar collagen (Chronopar, Chronolog, Inc.). The fibrillar coating method was detailed previously by Casa et al, where microfluidic channels were filled with a 100 μg/mL collagen type I solution in 0.9% saline and incubated in a humid environment at room temperature for 24 hours. The collagen-coated microfluidic chips were positioned on the stage of a light microscope (DM6000B, Leica Microsystems) with a 4× objective and connected to an upstream reservoir with Tygon tubing. Downstream tubing led to four discharge reservoirs, each placed on a precision balance (Ohaus Scout SPX222, Ohaus Corp) to measure mass flow rates. Maximum shear rates were calculated at 6500 s⁻¹ from experimental flow rates through the channel. Occlusion time, t_{occ}, was measured as the time from first blood contact in the stenosis region of the channel to the time of the initial maximum mass reading. The average and standard deviation of t_{occ} was calculated for each concentration of particles. Statistical analysis was performed between groups with a t-test (p-value<0.01).

### Results

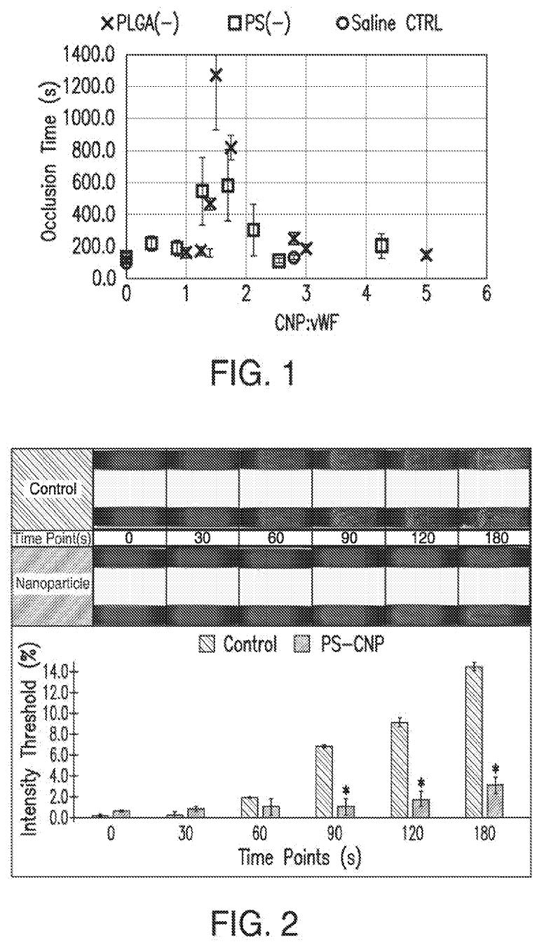

Experimental shear-induced platelet accumulation indicates a similar dose response of polystyrene (PS) and poly(lactic-co-glycolic acid) (PLGA) nanoparticles relative to vWF concentration (FIG. 1). Negatively charged particles of both materials were compared to controls with volumetric equivalent additions of saline (no particles).

### Example 2: Negatively Charged Particles Induces a Reduction in the Rate of Shear-Induced Platelet Accumulation

### Materials and Methods

Porcine whole blood was obtained from a local abattoir (Holifield Farms, Covington, Ga.) and lightly heparinized at 3.5 USP units/mL. Blood was stored at room temperature on a rocker prior to testing. All testing was completed within 8 hours after collection.

Blood samples were treated with a PLGA particle dose of 28 μg/mL blood as the test group, with a non-treated blood group utilized as the control.

Microfluidic chips were created by casting PDMS (Sylgard 184, Krayden) on a custom-etched silicon 3D stenotic microfluidic mold. After plasma bonding the PDMS to 25×75 mm glass slide, the devices were coated with bovine type I fibrillar collagen (Chronopar, Chronolog, Inc.). The fibrillar coating method was detailed previously by Casa et al, where microfluidic channels were filled with a 100 μ.g/mL collagen type I solution in 0.9% saline and incubated in a humid environment at room temperature for 24 hours. The collagen-coated microfluidic chips were positioned on the stage of a light microscope (DM6000B, Leica Microsystems) with a 4× objective. Images of thrombus formation were acquired every 500 ms with a high-resolution CCD camera (Pixelfly, PCO). Image acquisition was facilitated by the μManager open-source microscopy software. Light transmittance was calculated at varying time points using MATLAB.

### Results

An effective concentration of negatively charged particles showed a reduction in the rate of shear-induced platelet accumulation as compared to non-treated control groups, as seen in FIG. 2. Platelet accumulation was significantly delayed for the two microfluidic test sections treated with particles.

### Example 3: Computational Simulation of Charged Nanoparticle-vWF Interaction

In this section, we demonstrate the consequence of the interaction of one or more negatively charged particles with the vWF in shear flow. The results here are based on physical and mathematical modeling and analysis. Significant conformational changes occur even when one charged nanoparticle attaches to vWF. With zeta potential and concentration of charged nanoparticle and vWF used in experiments of Examples 1 and 2, we present a summary of the analysis based on physical principles.

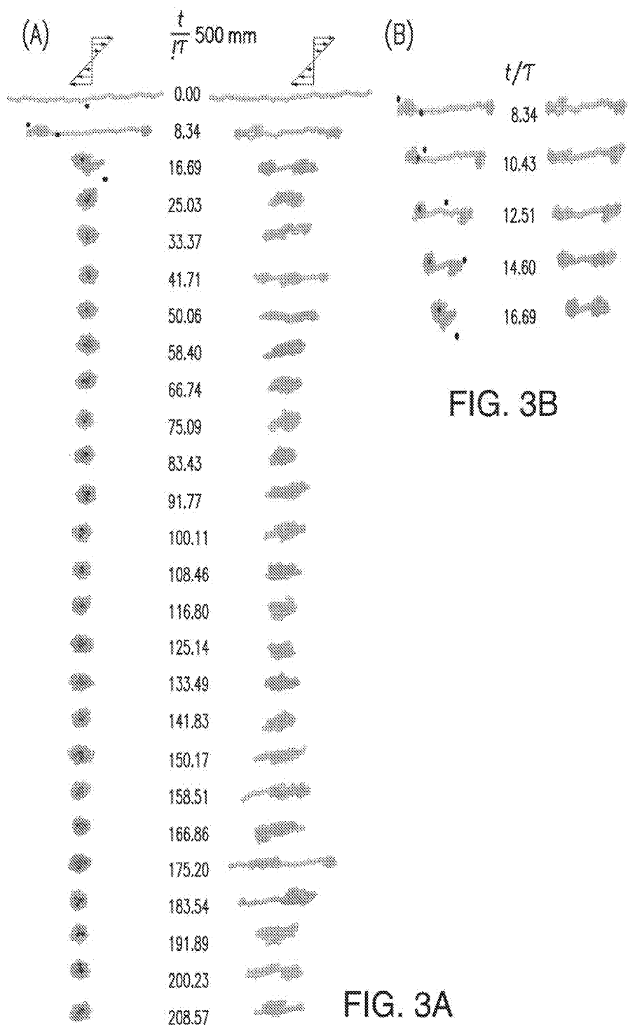

Based on this analysis, the relevant time scale here is the particle (monomer) Brownian diffusion time, τ. Several cases have been simulated where the ratio of the number of charged nanoparticles to the number of vWF is varied, as well as shear rate and the particles charge. Typical results at shear rate 6000 1/s are shown in FIG. 3. The initial condition is fully elongated vWF release in very dilute suspension of charged nanoparticles, all under shear flow at 6000 1/s. The particle charge is −50 mV. There is significant difference in the conformational dynamics of vWF with and without the charged nanoparticle. Note that nanoparticles with no charge, but otherwise identical size and concentration of charged particles, have no effect on vWF.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

1. A method for altering tertiary length of vWF comprising administering to a subject in need thereof a composition comprising negatively charged, non-functionalized polymeric particles in an amount effective to bind to vWF proteins and substantially change elongated vWF proteins into a globular state in the subject, wherein the globular vWF proteins are under physical shear stress and cannot bind to platelet receptors.

2. The method of claim 1, wherein altering the tertiary length of vWF extends or prolongs platelet occlusion time in a subject in need thereof.

3. The method of claim 1, wherein altering the tertiary length of vWF reduces or inhibits myocardial infarction or stroke in a subject in need thereof.

4. The method of claim 1, wherein altering the tertiary length of vWF inhibits or reduces the bioactivity of vWF in a subject in need thereof by inhibiting or reducing the interaction between the vWF proteins and platelets in the circulatory system of the subject.

5. A method for reducing or inhibiting shear-induced platelet accumulation in a subject in need thereof comprising administering to the subject an effective amount of negatively charged particles having an average diameter between 25 to 300 nm and a charge of −1 mV to −500 mV to bind to the A1 domain of vWF proteins in the circulatory system of the subject and inhibit or reduce binding of nanoparticle-bound-vWF proteins to platelets in the subject's circulatory system.

6. The method of claim 1, wherein binding of nanoparticles to the vWF proteins is electrostatic.

7. The method of claim 5, wherein the shear-induced platelet accumulation occurs in an artery or vein of the subject.

8. The method of claim 1, wherein the nanoparticles have an average diameter of about 10 to 1000 nm.

9. The method of claim 1, wherein the nanoparticles have an average diameter of between 25 and 300 nm.

10. The method of claim 1, wherein the nanoparticles comprise a biodegradable, negatively charged material.

11. The method of claim 10, wherein the negatively charged material is negatively charged under physiological conditions.

12. The method of claim 10, wherein the nanoparticles comprise poly(lactic-co-glycolic acid)

13. The method of claim 1, wherein the negatively charged nanoparticles have a charge of about −1 to −500 mV

13a. (canceled)

14. (canceled)

15. The method of claim 1, wherein the platelet receptors include Glycoprotein Ib or Glycoprotein IIb/IIIa (also known as Integrin α_{IIb}β₃).

16. The method of claim 1, wherein the elongation of vWF under high shear rates greater than 2000/s is inhibited.

17. A pharmaceutical composition comprising negatively charged polymeric nanoparticles formulated for parenteral administration, wherein the nanoparticles have an average diameter from 25 nm to 300 nm and a surface charge of between −25 mV and −80 mV.

18. The pharmaceutical composition of claim 17, wherein the nanoparticles are non-functionalized.

19. The pharmaceutical composition of claim 17, wherein the nanoparticles are not functionalized with a protein, lipid, therapeutic agent, or small molecule.

20. The pharmaceutical composition of claim 19, wherein the polymeric nanoparticles comprise poly(lactic-co-glycolic acid).

21. The method of claim 1, wherein the negatively charged nanoparticles have a charge of about −25 to −80 mV.
